(19) United States
(12) Patent Application Publication
(10) Pub. No.: US 2019/0237175 A1
(43) Pub. Date: Aug. 1, 2019

(54) SYSTEM FOR AUTOMATED ANALYSIS OF MCS LOG FILES

(71) Applicant: HeartWare, Inc., Miami Lakes, FL (US)

(72) Inventors: Michael C. BROWN, Dresher, PA (US); Carlos REYES, Davie, FL (US); Daniel TAMEZ, Plantation, FL (US); Neil VOSKOBOYNIKOV, Miami, FL (US)

(21) Appl. No.: 16/263,872

(22) Filed: Jan. 31, 2019

Related U.S. Application Data
(60) Provisional application No. 62/624,930, filed on Feb. 1, 2018.

(51) Int. Cl. G16H 15/00; A61M 1/10; G16H 80/00

(52) U.S. Cl. CPC G16H 15/00; A61M 2205/3584; G16H 80/00; A61M 1/1086

(57) ABSTRACT

A method of filtering log file data from a log file of an implantable blood pump, the method including receiving the log file from a controller coupled to the implantable blood pump, automatically filtering the log file to differentiate an urgent data file from a routine data file, and generating an urgent report displaying the urgent data file and a routine report displaying the routine data file within a rapid time period.

### CROSS-REFERENCE TO RELATED APPLICATION

This application claims the benefit of U.S. Application Ser. No. 62/624,930, filed Feb. 1, 2018.

### FIELD

This disclosure relates to a method and system for automatically analyzing a log file associated with an implantable blood pump for immediate clinical review.

### BACKGROUND

Mechanical Circulatory Support Devices (“MCSDs”) are lifesaving mechanical devices configured to assist the heart in pumping blood throughout the body. A known type of MCSD is a ventricular assist device (“VAD”) which may include a centrifugal pump, axial pump, or another type of electromagnetic pump configured to pump blood from the heart to the rest of the body. One such centrifugal pump is the HVAD® Pump and one such axial pump is the MVAD® Pump, each manufactured by HeartWare, Inc. in Miami Lakes, Fla., USA.

VADs are typically controlled using a controller configured to store one or more types of log files. For example, known controllers often store data, alarm, and event log files. The log files may be downloaded, such as when the patient having the implanted VAD visits a healthcare facility. Once downloaded, the files may be sent to a remote location for analysis, after which one or more reports are generated and returned to the healthcare facility and/or patient. As a drawback, when the log files are received by the remote location for analysis, the log files typically enter into a queue until the log files can be reviewed by one or more personnel at the remote location. The log files can be marked as “routine” or “urgent.” Urgent logs will generally undergo analysis within two hours, while routine logs will generally undergo analysis within eight hours. Such relatively long turn-around time may increase the health risks for the patient. In addition, such system may fail to identify relevant and useful urgent information.

### SUMMARY

The techniques of this disclosure generally relate to a method and system of analyzing log files associated with an implantable blood pump and rapidly generating a report.

In one aspect, the present disclosure provides a method of automatically analyzing log file data from a log file of an implantable blood pump. The method includes receiving the log file from a controller coupled to the implantable blood pump. The log file is automatically analyzed. Data from the log file is automatically extracted. A report is generated displaying the extracted data within a period of time between one to ten minutes.

In another aspect, the extracted data includes a plurality of blood pump parameters.

In another aspect, the method further includes determining a plurality of expected blood pump parameters and using a blood pump trend analysis to compare the plurality of expected blood pump parameters to a plurality of blood pump parameters from the log file.

In another aspect, the log files include a data log file, an alarm log file, and an event log file.

In another aspect, the method further includes dividing the report into a plurality of sections, each of the plurality of sections including information extracted from one from the group consisting of the data log file, the alarm log file, and the event log file.

In another aspect, the method further includes transmitting the report to a clinician in a remote location.

In another aspect, the generated report includes a plurality of patient parameters, the plurality of patient parameters including at least one of a group consisting of a heart rate trend, a heart rate variability trend, and an aortic status trend.

In another aspect, the generated report includes a plurality of waveforms each corresponding to a blood pump parameter.

In another aspect, the generated report includes a highlighted region displaying a normal pulsatility graph and an abnormal pulsatility graph.

In another aspect, the method further includes generating the report within one to five minutes after receipt of the log file.

In another aspect, the method further included automatically analyzing a plurality of pump parameters. A pump parameter trend analysis is generated using the plurality of pump parameters. a circadian cycle is determined using the pump parameter trend analysis.

In one aspect, a method of analyzing a plurality of log files of an implantable blood pump includes receiving the plurality of log files from a controller coupled to the implantable blood pump. The plurality of log files is automatically analyzed and divided into a plurality of data types. A report displaying a plurality of pump parameters associated with at least one of the plurality of data types is automatically generated. The report is transmitted to a user for review within a time period between one to ten minutes after receipt of the plurality of log files.

In another aspect, the method further includes automatically generating the report within one to five minutes.

In another aspect, the method further includes graphically plotting the plurality of pump parameters using a plurality of waveforms and isolating a portion of at least one of the plurality of waveforms.

In another aspect, the report includes an alarm indicator and an event indicator.

In one aspect, a system for automatically analyzing log file data from a log file of an implantable blood pump includes a remote device coupled to a controller in communication with an implantable blood pump. The remote device is configured to receive a log file from a controller coupled to the implantable blood pump and automatically analyze and extract data from the log file. The remote device generates a report displaying the extracted data file within a period of time between one to ten minutes.

In another aspect, the report includes a plurality of pump parameters displaying using a plurality of waveforms.

In another aspect, the extracted data includes a plurality of blood pump parameters.

In another aspect, the remote device is further configured to determine a plurality of expected blood pump parameters and use a blood pump trend analysis, compare the plurality of expected blood pump parameters to a plurality of blood pump parameters from the log file.

In another aspect, the log files include a data log file, an alarm log file, and an event log file.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a block diagram depicting an exemplary system for operating an implantable blood pump constructed in accordance with the principles of the present application;

FIG. 2 is a flow chart showing an exemplary method of analyzing a log file and extracting data from the log files in accordance with the principles of the present application;

FIG. 3 is a block diagram showing communication between a controller and remote device of the present application;

FIG. 4 shows an exemplary report page showing pump parameter trends plotted on a graph having flow and power values, speed values, and data and time stamps;

FIG. 5 shows an exemplary report page displaying an exemplary trend analysis, the patient information section, and the pump parameter information section;

FIG. 6 shows an exemplary report page including a highlighted region displaying a normal pulsatility graph indicating the absence of a suction condition and an abnormal pulsatility graph indicating a presence of a suction condition;

FIG. 7 shows an exemplary report page including a power consumption summary section;

FIG. 8 shows an exemplary report page including an additional notes section and a battery summary section;

FIG. 9 shows an exemplary section of a report page illustrating the number of times an alarm within the blood pump was triggered during the 14-day time period;

FIG. 10 shows an exemplary report page displaying a trend report of pump parameters, a heart rate section containing a waveform, a daily aortic valve opening section, a circadian cycle section, and a suction burden percentage section;

FIG. 11 shows an exemplary report page including a circadian cycle section including a graphical depiction of the flow waveform and the speed waveform over an eight-day period; and

FIG. 12 shows an exemplary report page including a custom zoom feature allowing a clinician to select a time period windows to view pump parameters.

### DETAILED DESCRIPTION

Before describing in detail exemplary embodiments, it is noted that the embodiments reside primarily in combinations of apparatus components and processing steps related to automatically filtering log file data from a log file of an implantable blood pump. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

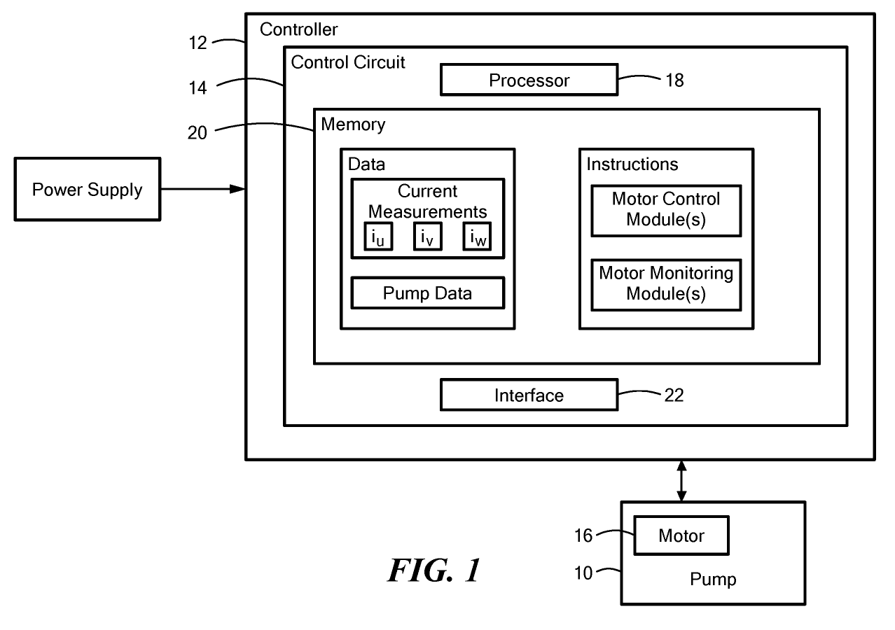

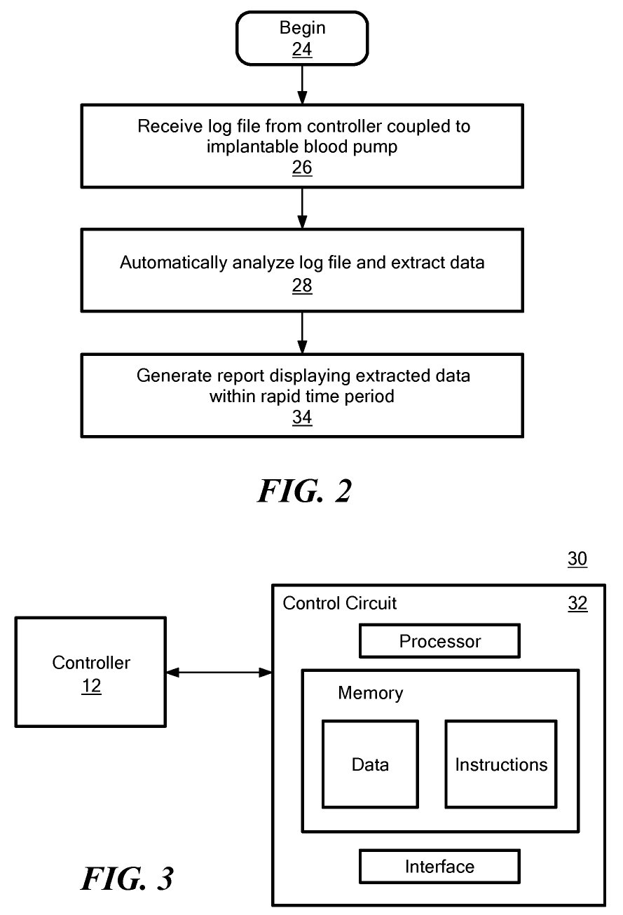

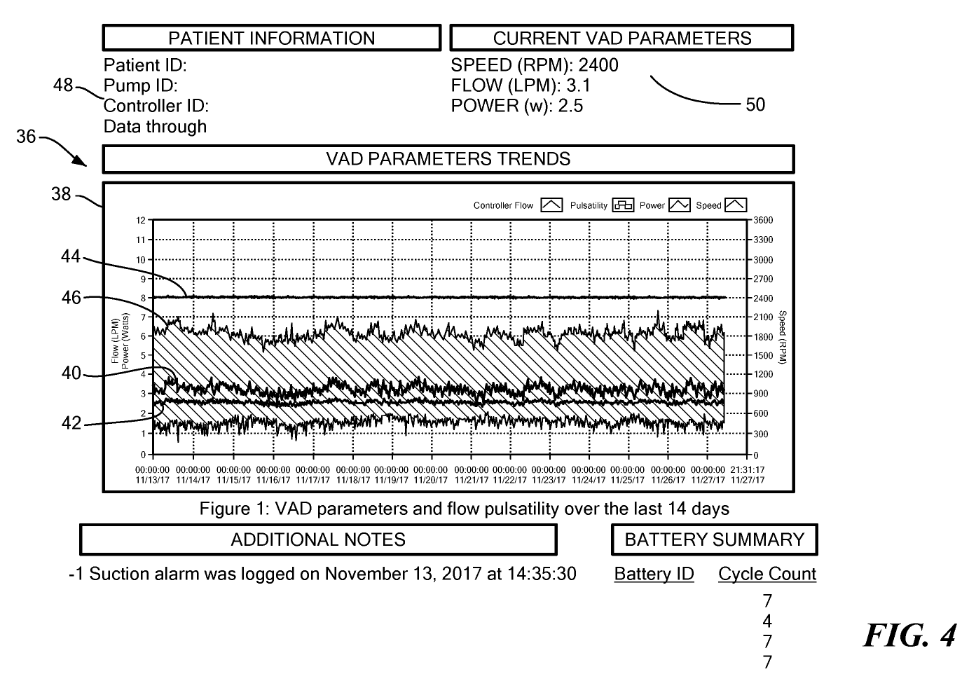

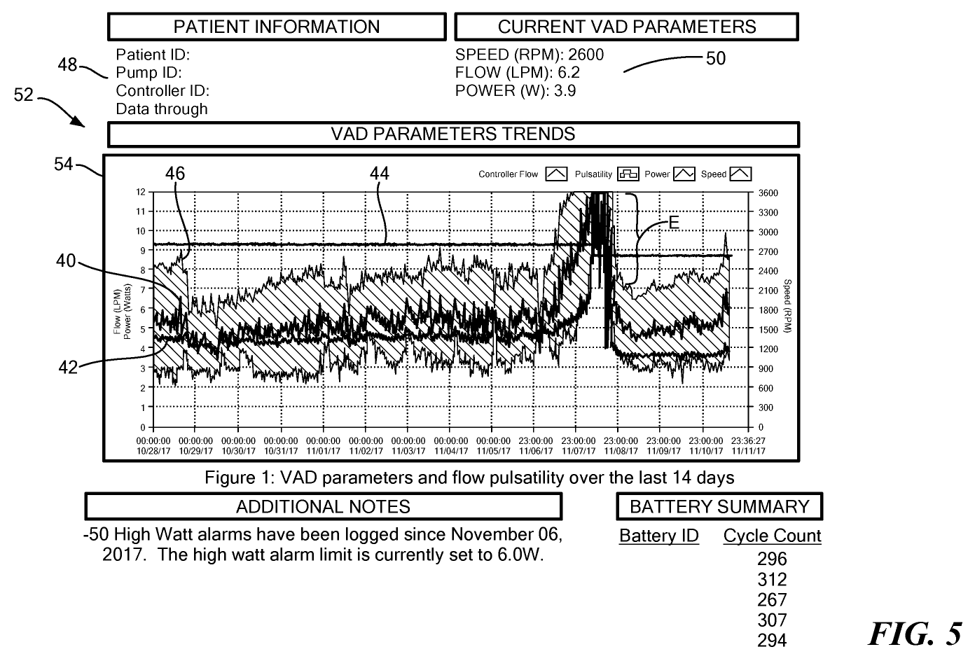

Referring now to the drawings in which like reference designators refer to like elements there is shown in FIG. 1 a block diagram depicting an exemplary system for operating the implantable blood pump, the system including an exemplary implantable blood pump generally designated as “10.” The blood pump 10 is configured to be entirely or partially implanted within a patient, such as a human or animal patient. The blood pump 10 may be, without limitation, the HVAD® Pump or the MVAD® Pump, having a movable element, such as a rotor, configured to pump blood from the heart to the rest of the body. The HVAD® Pump is further discussed in U.S. Pat. Nos. 7,997,854 and 8,512,013, the disclosures of which are incorporated herein by reference in the entirety. The MVAD® Pump is further discussed in U.S. Pat. Nos. 8,007,254, 8,419,609, and 9,561,313, the disclosures of which are incorporated herein by reference in the entirety. The system includes a controller 12 in communication with the blood pump 10 which may be positioned external to or implanted within the patient.

In one configuration, the controller 12 may be configured to determine, monitor, and/or track one or more blood pump parameters and store the blood pump parameters and associated patient information in one or more log files. In one configuration, the log files include a data log file, an alarm log file, and event log file; however, other types of log files may be included.

The information in the data log file may include pump parameters, such as pump speed, power usage or consumption, flow, electrical current, voltage, and/or back electromotive force (“EMF”). Further details associated with methods of determining current, voltage, and EMF within a blood pump, are disclosed in commonly owned U.S. Pat. No. 9,511,179, which is hereby incorporated by reference in the entirety. In one configuration, information in the data log file may be recorded every fifteen minutes. In other configurations, the data log file may be recorded more frequently, such as every 5 minutes, every minute, or the like. When plotted versus time, the data log file information may be used to observe trends in the pump parameters and identify adverse events. For example, fluctuations in power usage may indicate a presence of an adverse event, such as thrombus. Other adverse events which may be identified by analyzing pump parameters include ingestion, GI bleed, occlusion, and the like.

In one configuration, the alarm log files contain entries recorded at the onset of an alarm, such as an alarm in communication with the blood pump. The event log files may store information about various events, such as changes to the blood pump's controller settings, for example speed changes, hematocrit changes, and/or alarm limit changes and suction. Entries may be recorded in the event log files at the time of a particular event.

As shown in FIG. 1, the controller 12 includes a control circuit 14 configured to monitor and control startup and subsequent operation of a motor 16 implanted within the blood pump 10. The controller 12 may also include a processor 18, a memory 20, and an interface 22. The memory 20 may be configured to store information accessible by the processor 18, including instructions executable by the processor 18 and/or data, such as the log files, that may be retrieved, manipulated or stored by the processor 18. Further details associated with an exemplary controller 12 are disclosed in commonly owned U.S. patent application Ser. No. 15/710,323, which is hereby incorporated by reference in the entirety.

With reference to FIG. 2, numerous method steps are depicted which may be used to analyze the log file and extract data from the log files in accordance with the method disclosed herein. One or more steps may be added and/or omitted and the order of the steps may differ from that which is shown. In one configuration the method begins at step 24 and proceeds to step 26 of receiving one or more of the log files, such as the data, event, and alarm log files, from the controller 12. In step 28, the method may include automatically analyzing the log file and extracting log file data. In other words, the method may include a system for automatically analyzing the log file to identify and divide the log files into one or more data types or categories of information.

For example, with reference to FIG. 3, the log files may be wired or wirelessly transmitted from the controller 12 to a system 30 including a remote device 32 configured to download the log files, analyze the log files, and extract data. The remote device 32 may be located within a clinician's office, a treatment center, a patient monitoring station, or the like, and may include a control circuit, a remote processor, a memory, a data storage unit, an instructions unit, a user interface, and/or a display screen. In one exemplary configuration, the remote device 32 may be configured to analyze the log files line by line, remove duplicate entries, and interleave and sort the log files using timestamps. In one example, the duplicate entries are identified by searching for repeat individual controller identification and sequence identification combinations. Once located, one or more data lines containing the repeat information may be deleted.

With reference to FIG. 2, in step 34, one or more reports may be generated displaying the extracted data within a rapid time period, such as one to ten minutes or one to five minutes after receipt of the log file, or less than a minute. The generated reports or reports may be in electronic or printed format. As such, the reports may be immediately viewable in real-time on a display screen when the system 30 is located within the clinician's office. In the alternative, such as when the system 30 is located within a patient monitoring station, the reports may be immediately transmitted to the clinician in the clinician's office or another remote location. The rapid time period provides the clinician with information associated with the blood pump and patient as supplemental information to clinical presentation relatively quickly, which may be beneficial in emergency situations, to reduce the patient's waiting time, and the like.

In one configuration, the report includes information associated with an adverse event, the blood pump's system performance, and/or the blood pump's system operation. For example, the report may alert the clinician to one or more circumstances that require immediate attention or may indicate that the blood pump is operating within a normal range for the particular blood pump and patient.

In order to provide for relatively organized viewing, the method may include the system 30 dividing the report into one or more sections, with each section including information extracted from the data log file, the alarm log file, or the event log file. In other words, information within each type of log file may be used to populate a corresponding section of the report. In one configuration, each report may have the same sections including numerous pages displaying the extracted data in various formats. In other configurations, the reports may contain select sections.

For example, FIG. 4 depicts an exemplary report section 36 or page as part of the report. The report section 36 is shown displaying an exemplary extracted data file filtered by the system 30. More specifically, the system 30 may automatically analyzing the pump parameters stored in the log file and use the pump parameter information to generate a pump parameter or blood pump trend analysis 38 in the form of a graph including one or more waveforms. The trend analysis may be used, for example, to determine and/or analyze expected pump parameters and detect changes relative thereto.

FIG. 4 depicts the pump parameter trends plotted using the graph having flow and power values, speed values, and data and time stamps. The pump parameters of FIG. 4 include a flow waveform 40, a power waveform 42, and a speed waveform 44 recorded over a time period, such as 14 days. In addition, a pulsatility waveform 46 is shown including peak and trough values. In one configuration, the exemplary report section 36 may contain a patient information section 48 and a pump parameter information section 50 populated by the system using a data point, such as a last data point from the log file.

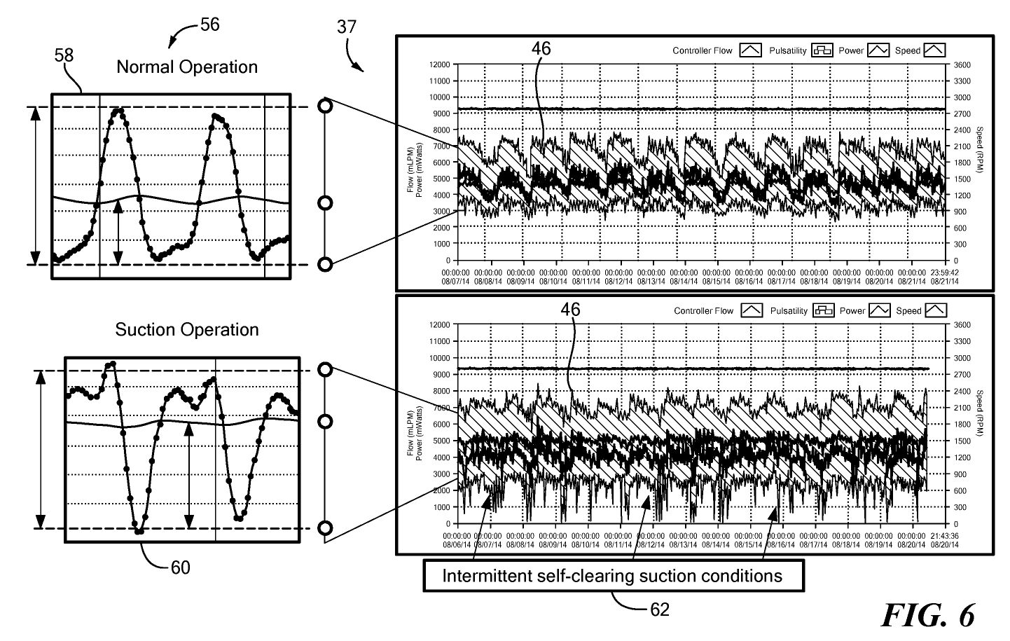

Referring now to FIG. 5, an exemplary report section 52 is provided displaying an exemplary trend analysis 54, the patient information section 48, and the pump parameter information section 50. In one configuration, the system 30 may be configured to compare the expected pump parameters to the log files and produce a visual depiction of the pump parameters exceeding the expected pump parameters by a threshold. The expected pump parameters may also be compared to the pump parameters measured in real-time. The threshold may vary in accordance with individual patents and the individual pump parameters; however, generally includes a region which signifies a health risk for the patient. For example, as shown in FIG. 5, an increase in the flow waveform 40, the power waveform 42, and the pulsatility waveform 46 relative to the expected pump parameters or baseline are depicted within a region generally designated as “E” which may signify an adverse event or health risk for the patient.

Referring now to FIG. 6, the method may include isolating a portion of at least one of the waveforms. For example, FIG. 6 depicts another exemplary report section 37 including a highlighted region 56 displaying a normal pulsatility graph 58 indicating the absence of a suction condition and an abnormal pulsatility graph 60 indicating a presence of a suction condition. In one configuration, the abnormal pulsatility graph 60 includes one or more indicators 62 identifying a fall in the pulsatility waveform 46 relative to normal operation. The characteristics of the pulsatility waveform 46 may be used to identify the suction condition as intermittent and self-clearing, thus signifying a non-emergency situation.

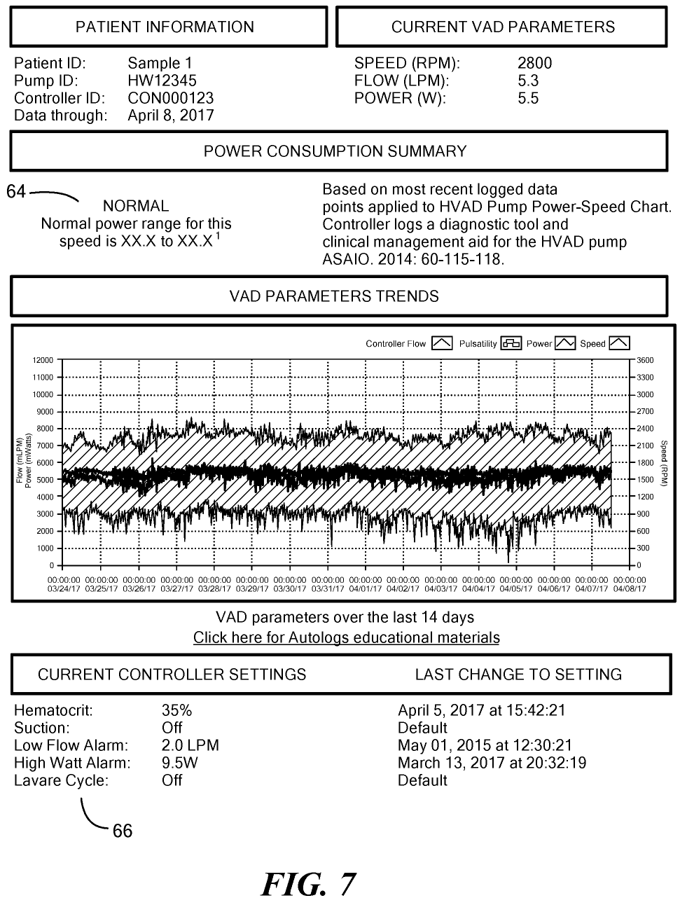

With reference to FIG. 7, the report may include a power consumption summary section 64 which may be populated by the system 30 using the last plotted value for speed and power in the data log file and comparing such values to a range of expected power. The range of expected power may be obtained through measurements or data, such as existing chart data provided by one or more test reports. In one configuration, the system 30 is configured to populate a current controller settings section 66 using the event log file. More specifically, the system 30 may be configured to analyze the controller setting entries over a select duration which may be outside of the 14-day time period. For example, the clinician may select a desired report section 36 between a preselected range of 3 days, 7 days, 14 days, 30 days, 60 days, or 90 days, or a custom range selected by the clinician.

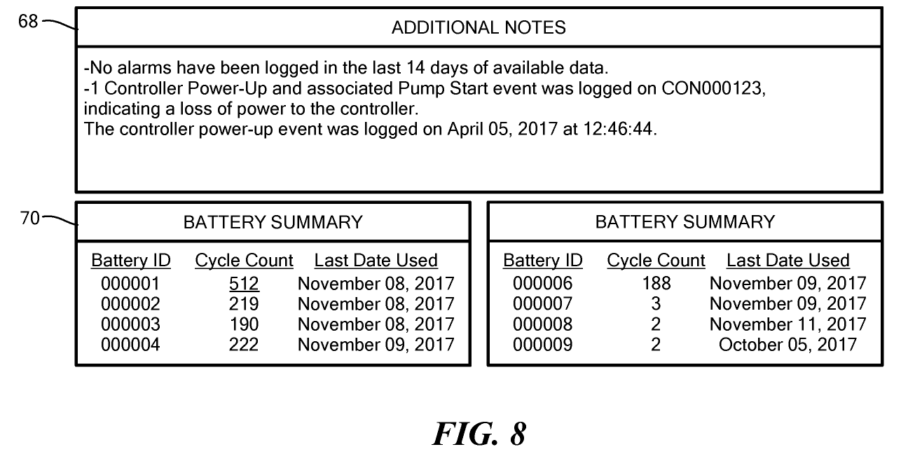

Referring now to FIG. 8, in one configuration, the report may contain an additional notes section 68 and a battery summary section 70. The addition notes section 68 section may be populated using relevant entries in the alarm log file and the event log file during, for example, a 14-day time period, whereas the is battery summary section 70 may be populated by considering the majority or all of the battery related entries in the data file. Each battery may contain a unique identification number used to report a maximum logged cycle count and a date of most recent use. For example, values over 500 cycles are considered outside of a useful lifespan and thus may be displayed in a designated font, such as a red font.

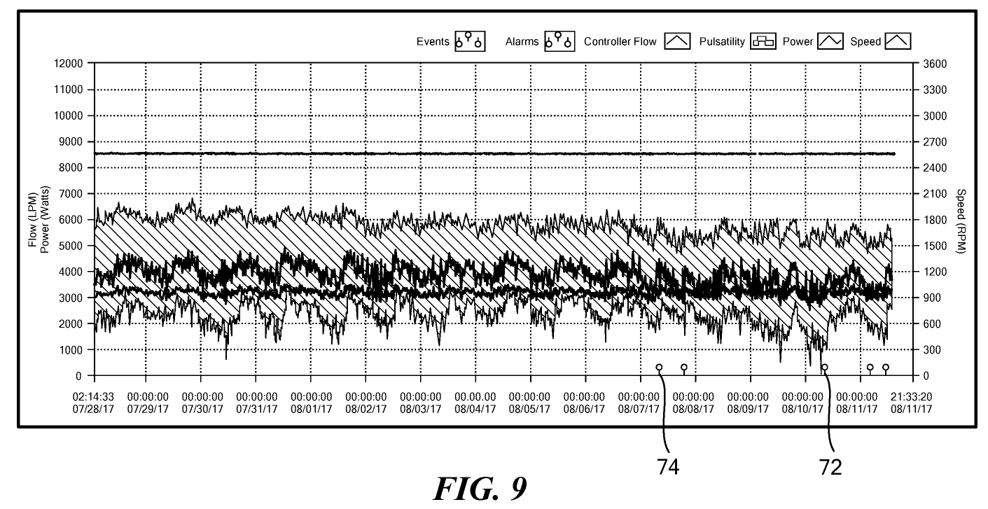

With reference to FIG. 9, in one configuration, the system 30 may be configured to generate the report to include an alarm indicator 72 and/or an event indicator 74. For example, the alarm indicator 72 shown in FIG. 9 illustrates the number of times an alarm within the blood pump 10 was triggered during the 14-day time period. In another example, the log files may include recorded information reflecting that 10 low flow alarms occurred and the corresponding report may include an indicator, such as a red symbol, of where the low flow alarms occurred in relation to the trend analysis. The event indicator 74 may be configured to indicate the number of times a designated event occurred within, for example, a 14-day time period. A user may input selection criteria to trigger the activation of the event indicator 74. For example, the user may choose to indicate on the trend analysis that the controller speed or hematocrit was adjusted multiple times using the event indicator, such as a blue symbol.

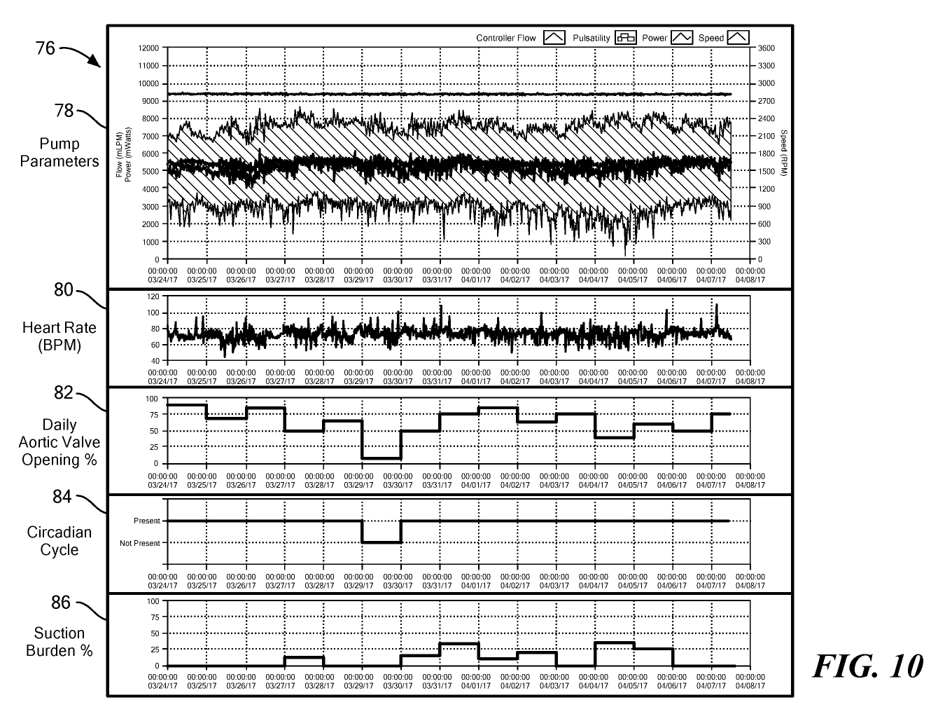

Referring now to FIG. 10, the report may include a set of patient criteria or patient parameters, such as a heart rate trend, a heart rate variability trend, an aortic status trend, and the like. For example, FIG. 10 depicts an exemplary report section 76 displaying a trend report of pump parameters 78, a heart rate section 80 containing a waveform, a daily aortic valve opening section 82, a circadian cycle section 84, and a suction burden percentage section 86.

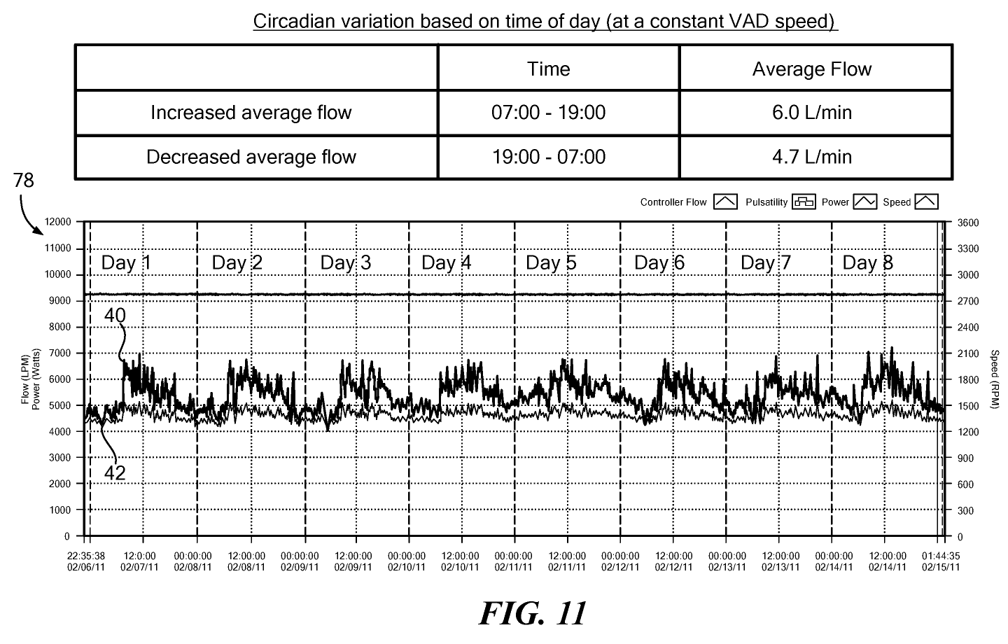

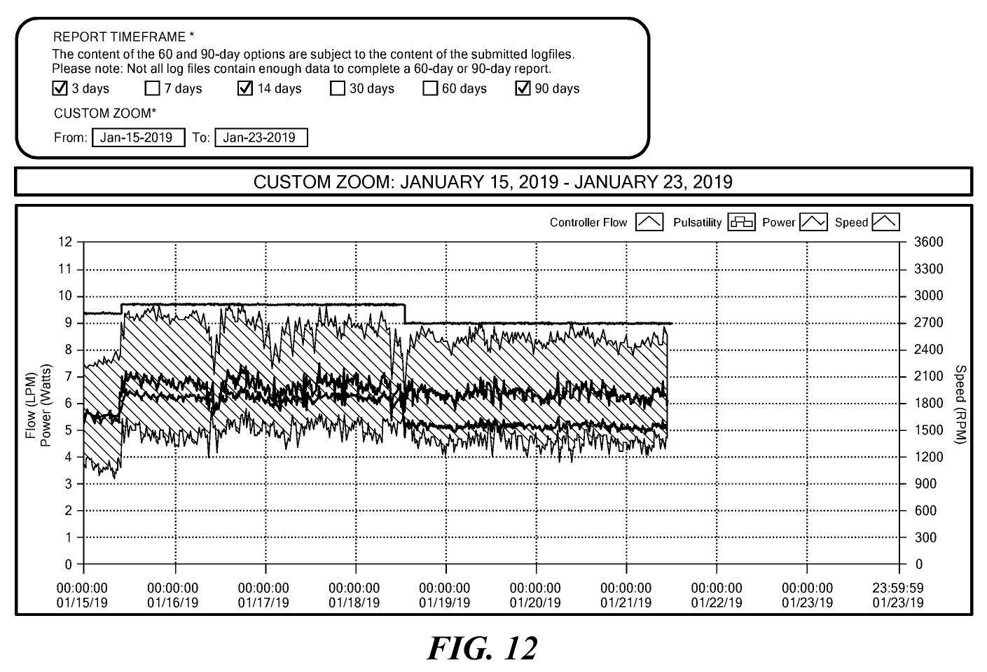

In one configuration, with reference to FIG. 11, the system 30 may be configured to analyze the pump parameter trend data from the log file, such as flow and speed data, and identify or determine the patient's circadian cycle. Information associated with the circadian cycle may be provided in a circadian cycle section 88 and may include a graphical depiction of the flow waveform 40 and the speed waveform 44 over a time period, such as 7 to 8 days.

Referring now to FIG. 12, in one configuration, the system 30 may be configured to analyze the controller setting entries over a select duration which may be outside of the 14-day time period. For example, the clinician may select a desired report section 36 between a preselected range of 3 days, 7 days, 14 days, 30 days, 60 days, and/or 90 days, or a custom range selected by the clinician. The custom range, shown as a “custom zoom” in FIG. 12 allows the clinician to isolate a particular report range time frame.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A method of automatically analyzing log file data from a log file of an implantable blood pump, the method comprising:
receiving the log file from a controller coupled to the implantable blood pump;
automatically analyzing the log file;
automatically extracting data from the log file; and
generating a report displaying the extracted data within a period of time between one to ten minutes.

2. The method of claim 1, wherein the extracted data includes a plurality of blood pump parameters.

3. The method of claim 2, further comprising:
determining a plurality of expected blood pump parameters; and
using a blood pump trend analysis to compare the plurality of expected blood pump parameters to a plurality of blood pump parameters from the log file.

4. The method of claim 1, wherein the log files include a data log file, an alarm log file, and an event log file.

5. The method of claim 4, further comprising dividing the report into a plurality of sections, each of the plurality of sections including information extracted from one from the group consisting of the data log file, the alarm log file, and the event log file.

6. The method of claim 1, further comprising transmitting the report to a clinician in a remote location.

7. The method of claim 1, wherein the generated report includes a plurality of patient parameters, the plurality of patient parameters including at least one of a group consisting of a heart rate trend, a heart rate variability trend, and an aortic status trend.

8. The method of claim 1, wherein the generated report includes a plurality of waveforms each corresponding to a blood pump parameter.

9. The method of claim 1, wherein the generated report includes a highlighted region displaying a normal pulsatility graph and an abnormal pulsatility graph.

10. The method of claim 1, further comprising generating the report within one to five minutes after receipt of the log file.

11. The method of claim 1, further comprising:
automatically analyzing a plurality of pump parameters;
generating a pump parameter trend analysis using the plurality of pump parameters; and
determining a circadian cycle using the pump parameter trend analysis.

12. A method of analyzing a plurality of log files of an implantable blood pump, the method comprising:
receiving the plurality of log files from a controller coupled to the implantable blood pump;
automatically analyzing and dividing the plurality of log files into a plurality of data types;
automatically generating a report displaying a plurality of pump parameters associated with at least one of the plurality of data types; and
transmitting the report to a user for review within a time period between one to ten minutes after receipt of the plurality of log files.

13. The method of claim 12, further comprising automatically generating the report within one to five minutes.

14. The method of claim 12, further comprising graphically plotting the plurality of pump parameters using a plurality of waveforms and isolating a portion of at least one of the plurality of waveforms.

15. The method of claim 12, wherein the report includes an alarm indicator and an event indicator.

16. A system for automatically analyzing log file data from a log file of an implantable blood pump, the system comprising:
a remote device coupled to a controller in communication with an implantable blood pump, the remote device being configured to:
receive a log file from a controller coupled to the implantable blood pump;
automatically analyze and extract data from the log file; and
generate a report displaying the extracted data file within a period of time between one to ten minutes.

17. The system of claim 16, wherein the report includes a plurality of pump parameters displaying using a plurality of waveforms.

18. The system of claim 16, wherein the extracted data includes a plurality of blood pump parameters.

19. The system of claim 18, wherein the remote device is further configured to:
determine a plurality of expected blood pump parameters; and
use a blood pump trend analysis, compare the plurality of expected blood pump parameters to a plurality of blood pump parameters from the log file.

20. The system of claim 1, wherein the log files include a data log file, an alarm log file, and an event log file.
